(19) United States
(12) Patent Application Publication
(10) Pub. No.: US 2024/0304290 A1
(43) Pub. Date: Sep. 12, 2024

(54) SYNTHETIC PATIENT DATA GENERATION

(71) Applicant: Hill-Rom Services, Inc., Batesville, IN (US)

(72) Inventors: Harsh Dweep, Buffalo Grove, IL (US); Christopher Nelson, Longmont, CO (US)

(21) Appl. No.: 18/587,485

(22) Filed: Feb. 26, 2024

Related U.S. Application Data
(60) Provisional application No. 63/489,332, filed on Mar. 9, 2023.

(51) Int. Cl. G16H 10/60

(52) U.S. Cl. CPC G16H 10/60

(57) ABSTRACT

A system is disclosed for generating synthetic patient data. The system includes one or more patient data monitoring devices, and a computing device that receives patient data from the one or more patient data monitoring devices. The system receives patient data from the one or more patient data monitoring devices. The system analyzes the patient data in real-time to learn one or more characteristics. The system generates synthetic patient data based on the one or more characteristics. The synthetic patient data including data representative of the patient data that is different from the patient data. The system stores the synthetic patient data in a database.

### BACKGROUND

To develop accurate algorithm models, enormous quantities of data pertaining to inputs of the algorithm models are typically needed. Unfortunately, many barriers exist for collecting data to develop algorithm models for medical devices in a hospital environment, such as privacy concerns, regulatory standards, and data ownership.

In some cases, these barriers can be overcome by initiating a clinical study which allows developers of algorithm models to collect the data needed within the hospital environment while meeting the previously mentioned concerns. However, the cost of such studies can be expensive and must often be repeated to develop a robust algorithm model.

### SUMMARY

In general terms, the present disclosure relates to creating synthetic patient data for medical device-oriented algorithm development while mitigating privacy, regulatory, and data ownership concerns. Various aspects are described in this disclosure, which include, but are not limited to, the following aspects.

One aspect relates to a system for generating synthetic patient data, the system comprising: one or more patient data monitoring devices; and a computing device that receives patient data from the one or more patient data monitoring devices, the computing device including: at least one processing device; and at least one computer readable data storage device storing software instructions that, when executed by the at least one processing device, cause the at least one processing device to: receive patient data from the one or more patient data monitoring devices; analyze the patient data in real-time to learn one or more characteristics; generate synthetic patient data based on the one or more characteristics, the synthetic patient data including data representative of the patient data that is different from the patient data; and store the synthetic patient data in a database.

Another aspect relates to a method for generating synthetic patient data, the method comprising: receiving patient data from one or more patient data monitoring devices; analyzing the patient data in real-time to learn one or more characteristics; generating synthetic patient data based on the one or more characteristics, the synthetic patient data including data representative of the patient data that is different from the patient data; and storing the synthetic patient data in a database.

Another aspect relates to a non-transitory computer readable storage media including computer readable instructions which, when read and executed by a computing device, cause the computing device to: receive patient data from one or more patient data monitoring devices; analyze the patient data in real-time to learn one or more characteristics; generate synthetic patient data based on the one or more characteristics, the synthetic patient data including data representative of the patient data that is different from the patient data; and store the synthetic patient data in a database.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 schematically illustrates an example of a system for generating synthetic patient data.

FIG. 2 is an isometric view of another example of a system for generating synthetic patient data.

FIG. 3 illustrates an example of a thermal image captured by a thermal camera of the system of FIG. 2.

FIG. 4 illustrates another example of a thermal image captured by the thermal camera of the system of FIG. 2.

FIG. 5 illustrates another example of a thermal image captured by the thermal camera of the system of FIG. 2.

FIG. 6 schematically illustrates an example of a method of generating synthetic patient data that can be performed by the systems of FIGS. 1 and 2.

FIG. 7 schematically illustrates an example of computing hardware components for implementing aspects of a computing device used in the systems of FIGS. 1 and 2.

### DETAILED DESCRIPTION

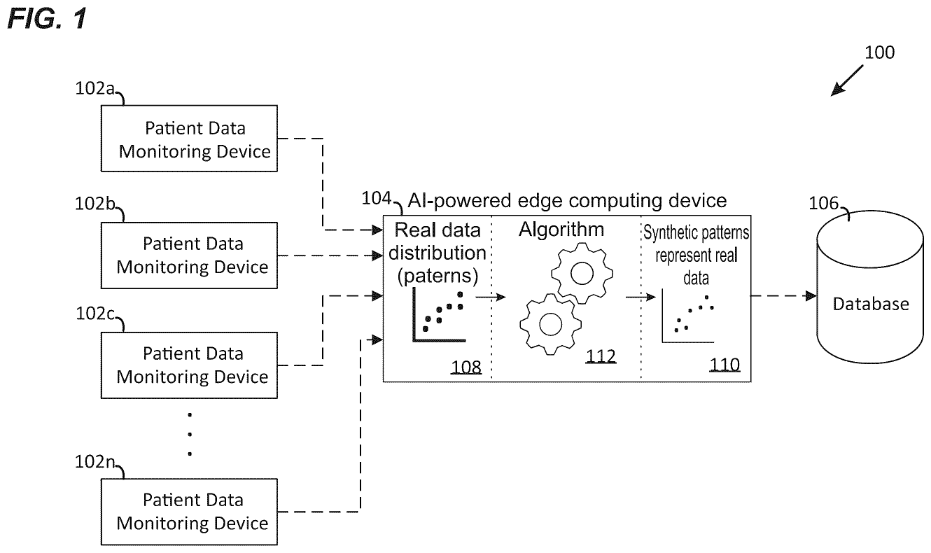

FIG. 1 schematically illustrates an example of a system 100 for generating synthetic patient data. The system 100 includes a computing device 104 that receives patient data 108 from a plurality of patient data monitoring devices 102a-102n. The computing device 104 learns features and patterns from the patient data 108, and transfers this information to a synthetic generating algorithm (SGA) 112 that generates synthetic patient data 110 without storing the patient data 108 received from the patient data monitoring devices 102a-102n.

The synthetic patient data 110 includes data point distributions, which represent the patient data 108 without including any actual data points from the patient data 108, thus protecting the privacy and the integrity of the patient data 108. For example, the patient data 108 can be fed into the SGA 112, and the SGA 112 outputs the synthetic patient data 110 which is similar to the patient data 108, but is different and does not exist in the real world.

As an illustrative example, the patient data 108 can include patient images which are fed into the SGA 112. The synthetic patient data 110 generated by the SGA 112 looks similar to the patient images of the patient data 108, but is different and does not exist in the real world.

As a further illustrative example, the patient data 108 can include patient positioning data that is inputted into the SGA 112. The synthetic patient data 110 generated by the SGA 112 is similar to the patient positioning data, but is different and does not exist in the real world. In this example, the synthetic patient data 110 is stored and may be used to train another algorithm, for example, an algorithm which monitors patient positioning and/or patient sleep patterns.

Illustrative examples of the SGA 112 can include, without limitation, generative adversarial networks (GANs), transformers, autoencoders, flow based generative models, and other types of artificial intelligence algorithms including machine learning frameworks/models.

In some examples, the computing device 104 includes an edge computing device that processes the patient data 108 in close proximity to the patient data monitoring devices 102a-102n without using a cloud server to improve response times and save bandwidth. This can also ensure that the privacy and integrity of the patient data 108 is maintained. The computing device 104 processes the patient data 108 in real-time to generate the synthetic patient data 110.

Once the synthetic patient data 110 is generated, the computing device 104 stores the synthetic patient data 110 in a database 106. This allows the synthetic patient data 110 to be used to develop one or more algorithms including artificial intelligence algorithms, including machine learning algorithms which are a subset of artificial intelligence algorithms. For example, the synthetic patient data 110 is configured for use as training data for building machine learning models instead of the actual patient data detected by the plurality of patient data monitoring devices 102a-102n. The synthetic patient data 110 that is output from the SGA 112 can be stored for later use, either alone or in combination with real world data, for creating new algorithms, improving existing algorithms, or generating new synthetic patient data.

The synthetic patient data 110 is not combined with the actual patient data, nor does it augment the actual patient data in the database 106. In this manner, the synthetic patient data 110 allows artificial intelligence algorithms to be developed, while maintaining the privacy of the patient data 108 collected by the patient data monitoring devices 102a-102n.

The patient data monitoring devices 102a-102n can include one or more sensors that measure physiological parameters of a patient. The one or more sensors can include any type of sensor including sensors that are implanted into the patient's body, sensors that are in contact with the patient's body (but are not implanted), and/or sensors that are contactless.

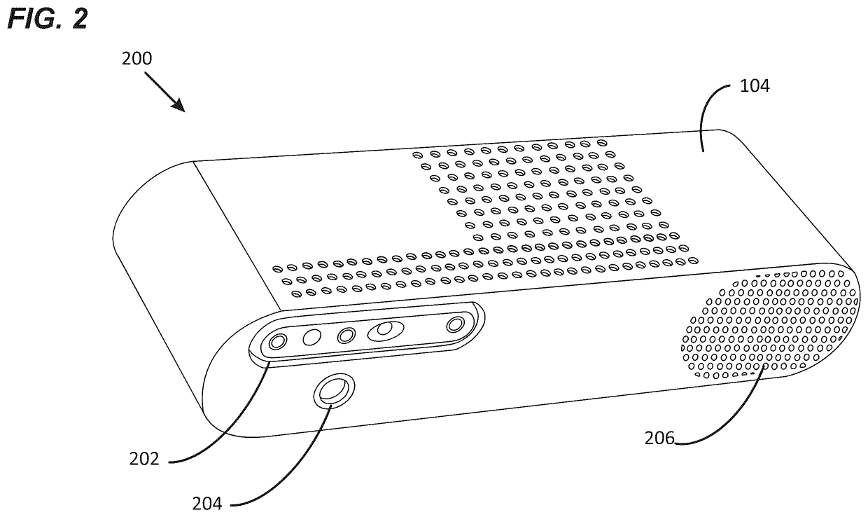

Illustrative examples of the patient data monitoring devices 102a-102n can include, but are not limited to: a camera that captures images and/or video streams of the patient; a pulse oximeter that indirectly monitors oxygen saturation (SpO2) of the patient's blood; a radar that detects one or more vital signs of the patient such as heart rate and respiration rate without contacting the patient's body; an electrocardiograph (ECG) that records the heart's electrical activity; a ballistocardiograph (BCG) device that measures ballistic forces generated by the heart by noninvasive methods from the surface of the patient's body; pressure sensors for capturing a body pressure map that measures the pressure distribution between the patient's body and a support surface such as a mattress of a hospital bed; sleep tracking sensors for measuring sleep patterns of the patient; a microphone for capturing patient audio (i.e., voice); laser and/or infrared light emitters; a thermal camera for capturing infrared thermography (IRT), thermal videos, and/or thermal imaging of the patient; fundus imagers and vision screeners; infusion pumps; hemodialysis and peritoneal dialysis systems; pain medicine delivery pumps; patient safety alarms; etc. In one example shown in FIG. 2, one or more of the patient data monitoring devices 102 are combined with the computing device 104 in a single device package.

FIG. 2 is an isometric view of another example of a system 200 for generating synthetic patient data. The system 200 is a multimodal system that incorporates one or more of patient data monitoring devices with the computing device 104 in a single device package. In this example, the system 200 includes an RGB-D camera 202 and a thermal camera 204. In further examples, the system 200 can include additional types of patient data monitoring devices. In the example shown in FIG. 2, the system 200 includes a microphone and speaker unit 206 for two-way communications between the patient and a remotely located caregiver.

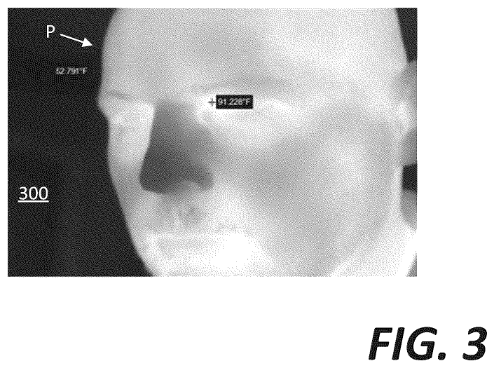

The RGB-D camera 202 is a type of camera that provides both depth (D) and color (RGB) data as output in real-time by providing pixel-to-pixel merging of RGB data and depth information to deliver both in a single frame. The thermal camera 204 captures infrared thermography (IRT), thermal videos, and/or thermal imaging of the patient, examples of which are shown in FIGS. 3-5. The images generated by the RGB-D camera 202 and the thermal camera 204 are examples of the patient data 108. As discussed above, the computing device 104 learns features and patterns from the patient data 108, and transfers this information to the SGA 112 that generates synthetic patient data 110 without storing the patient data 108 received from the RGB-D camera 202 and the thermal camera 204.

The system 200 is positioned such that the RGB-D camera 202 and the thermal camera 204 are in view of the patient. Like in the example of FIG. 1, the computing device 104 of the system 200 processes the patient data captured by the RGB-D camera 202 and/or the thermal camera 204 without using the computing capacity of a cloud server to improve response times and save bandwidth, and to also ensure that the privacy and integrity of the patient data is maintained. The computing device 104 processes the patient data captured by the RGB-D camera 202 and/or the thermal camera 204 in real-time to generate the synthetic patient data.

FIG. 3 illustrates an example of a thermal image 300 captured from a patient P by the thermal camera 204 of the system 200. The thermal images 300 includes patient data 108. Thermal images 300 can be continuously captured for continuously measuring the temperature of the patient P without contacting the patient. In this example, the temperature is measured from an area within the thermal image 300 such as the canthus of eye where the upper and lower eyelids meet. The system 200 can generate synthetic temperature measurements using the thermal image 300 without storing the thermal image 300 or the patient data 108 associated with the thermal image 300, and thereby protecting the privacy of the patient P.

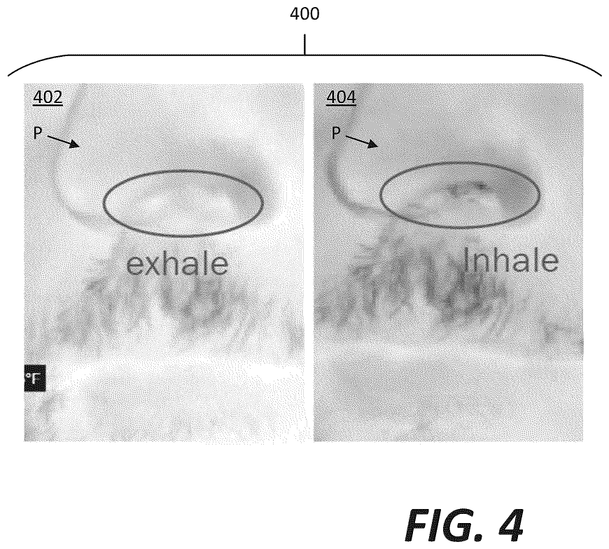

FIG. 4 illustrates another example of a thermal image 400 captured from the patient P by the thermal camera 204 of system 200. The thermal image 400 includes patient data 108. In this example, the thermal image 400 includes a first frame 402 captured when the patient P exhales, and a second frame 404 captured when the patient P inhales. Thus, the system 200 can use the thermal image 400 to continuously measure the respiration rate of the patient P without contacting the patient. The system 200 can generate synthetic respiration rate measurements using the thermal image 400 without storing the thermal image 400 or the patient data 108 associated with the thermal image 400, and thereby protecting the privacy of the patient P.

Additionally, the thermal images 300, 400 can be used by the system 200 to measure hemodynamics of the patient P (i.e., how blood flows through arteries and veins of the patient P) for predicting cardiovascular disease. The system 200 can generate synthetic hemodynamic data using the thermal images 300, 400 without storing the thermal images or the patient data 108 associated with the thermal images, and thereby protecting the privacy of the patient P.

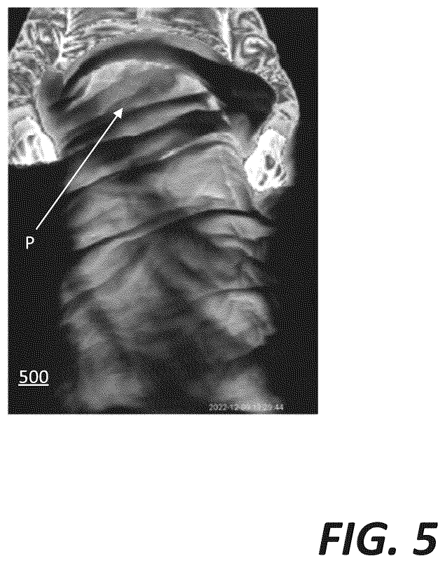

FIG. 5 illustrates another example of a thermal image 500 captured from the patient P by the thermal camera 204 of the system 200. In this example, the thermal image 500 can be used to continuously measure the position of the patient P when the patient P's body is at least partially obscured by one or more objects such as blankets. Thus, the thermal image 500 can be used by the system 200 to continuously measure the position of the patient P without contacting the patient. The system 200 can generate synthetic patient position data using the thermal image 500 without storing the original thermal image to protect the privacy of the patient P. In one example embodiment, the computing device 104 can generate synthetic images using the thermal image 500 streaming from the thermal camera 204.

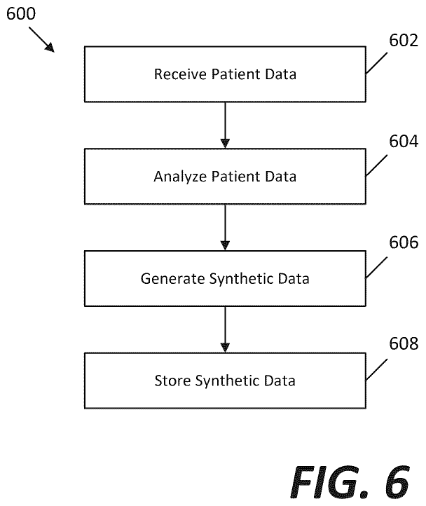

FIG. 6 schematically illustrates an example of a method 600 of generating synthetic patient data that can be performed by the computing device 104. The synthetic patient data can be used as training data for building one or more algorithms such as artificial intelligence and machine learning models. The synthetic patient data allows the one or more algorithms to be built while maintaining privacy of protected health information (PHI) associated with the patient data, and without requiring an ownership transfer and/or leasing of the patient data.

The method 600 includes an operation 602 of receiving patent data. In some examples, the patient data is received from the one or more patient data monitoring devices 102a-102n (see FIG. 1). In some examples, operation 602 includes receiving the patient data as images generated by the RGB-D camera 202 and the thermal camera 204 (see FIGS. 2-5).

Upon receiving the patient data, the method 600 includes an operation 604 of analyzing the patient data in real-time without storing the patient data. Operation 604 can include learning one or more characteristics such as features and/or patterns from the patient data.

Examples of characteristics that can be learned in operation 604 from video data of patients can vary. For example, the SGA 112 can learn to exclude feature(s) that identify the patient in the video data, and to include features such as a boundary of the patient (such as where the patient ends and the bed begins in the video data), and/or the location of one or more anatomical features of the patient such as the patient's eyes, head, arms, torso, hair, nose, mouth, and the like. The SGA 112 can also learn characteristics related to movement in the video data including, for example, getting out of bed, opening and closing of the patient's eyes, eating and drinking, chest movements during breathing, and other patient movements.

Characteristics can also be learned from audio data, which may include segmenting background noise from target audio, vocal patterns (such as waveforms, pitch, amplitude, volume, dialect, stuttering, etc.), and other types of audio. Characteristics can also be learned from non-communication audio such as coughing, snoring, and breathing sounds. Additionally, characteristics can be learned from non-human sounds such as alarms. Based on these types of data, the SGA 112 can create the synthetic patient data 110 such as images, video, or audio which do not copy the patient data 108 acquired from a real person such as a patient.

The method 600 includes an operation 606 of generating the synthetic patient data based on the patient data analyzed in operation 604. For example, operation 606 can include inputting the one or more characteristics such as the features and/or patterns learned in operation 604 into the SGA 112 to generate the synthetic patient data.

In one example embodiment, operation 606 can include generating synthetic patient data by inputting distributions obtained from radar based contactless sensors that measure one or more vital signs of the patient such as heart rate, respiration rate, and other vitals. In another example embodiment, patient data collected from a pulse oximeter is used to generate synthetic SpO2 waveforms. In another example embodiment, synthetic patient data can be generated from learning patient safety alarms and related aspects such as alarm nuisance and fatigue. In another example embodiment, operation 606 can include generating multimodal synthetic patient data sets by inputting patient data obtained from a plurality of sensors such as the RGB-D camera 202; the thermal camera 204; body wireframe data; audio (including voice recognition, natural language processing (NLP), sentiment evaluation, and the like); radar based contactless sensors; pulse oximeters; ECG; BCG; infrared, body pressure, and temperature maps; and the like.

Next, the method 600 includes an operation 608 of storing the synthetic patient data in the database 106. This allows the synthetic patient data to be used as training data for building one or more algorithm models including artificial intelligence and machine learning models. As an example, the synthetic patient data can be utilized to build improved algorithm models for early prediction of patient deterioration. As another example, the synthetic patient data can be utilized to build improved alarming algorithms such as for one or more types of medical devices.

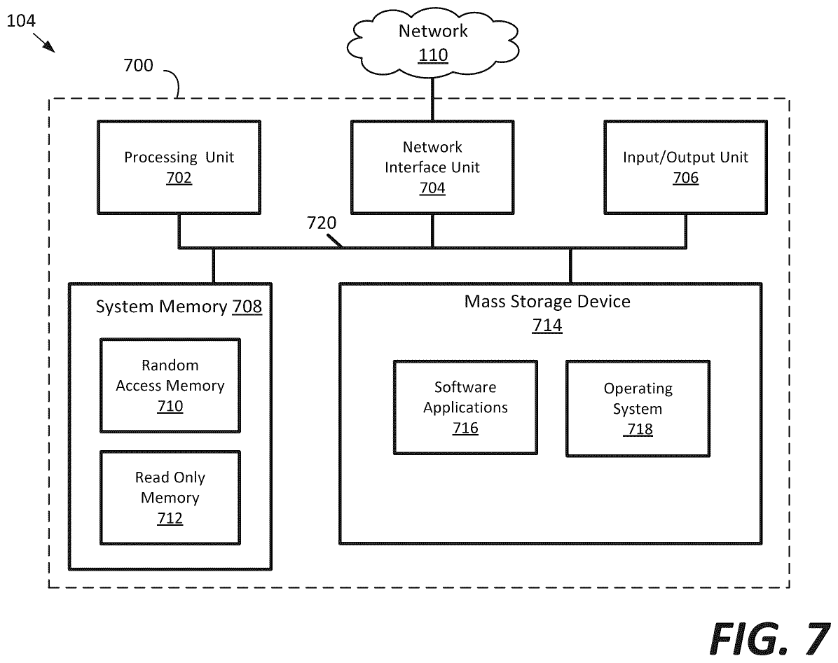

FIG. 7 schematically illustrates an example of computing hardware components 700 which can be used to implement aspects of the computing device 104. The computing hardware components 700 include a processing unit 702, a system memory 708, and a system bus 720 that couples the system memory 708 to the processing unit 702.

The processing unit 702 can include one or more processing devices such as a central processing unit (CPU). In some illustrative examples, the processing unit 702 includes one or more CPUs, microcontrollers, digital signal processors, field-programmable gate arrays, and/or other types of programmable electronic circuits.

The system memory 708 includes a random-access memory (“RAM”) 710 and a read-only memory (“ROM”) 712. A basic input/output logic containing the basic routines that help to transfer information between elements within the computing hardware components 700, such as during startup, is stored in the ROM 712.

The computing hardware components 700 can also include a mass storage device 714 that is able to store software instructions and data. The mass storage device 714 is connected to the processing unit 702 through the system bus 720. The mass storage device 714 and its associated computer-readable data storage media provide non-volatile, non-transitory computer-readable data storage for the computing hardware components 700.

Although the description of computer-readable data storage media contained herein refers to the mass storage device 714, it should be appreciated by those skilled in the art that computer-readable data storage media can be any available non-transitory, physical device or article of manufacture from which the device can read data and/or instructions. The mass storage device 714 is an example of a computer-readable storage device.

Computer-readable data storage media include volatile and non-volatile, removable, and non-removable media implemented in any method or technology for storage of information such as computer-readable software instructions, data structures, program modules, or other data. Example types of computer-readable data storage media include, but are not limited to, RAM, ROM, EPROM, EEPROM, flash memory, or other solid-state memory technology, or any other medium which can be used to store data for access by the computing device 104.

The computing hardware components 700 may operate in a networked environment using logical connections to remote network devices. For example, the computing hardware components 700 can connect to a communications network 120, such as a local area network, the Internet, or another type of computing network, through a network interface unit 704 connected to the system bus 720. The network interface unit 704 may also be utilized to connect to other types of networks and remote computing systems.

The computing hardware components 700 can also include an input/output controller 706 for receiving and processing input from a number of input devices. Similarly, the input/output controller 706 may provide output to a number of output devices.

The mass storage device 714 and/or the RAM 710 can be used store to software instructions 716, an operating system 718, and data for controlling the operation of the computing device 104. The software instructions 716, when executed by the processing unit 702, cause the computing hardware components 700 to perform the functions described herein.

The various embodiments described above are provided by way of illustration only and should not be construed to be limiting in any way. Various modifications can be made to the embodiments described above without departing from the true spirit and scope of the disclosure.

What is claimed is:

1. A system for generating synthetic patient data, the system comprising:
one or more patient data monitoring devices; and
a computing device that receives patient data from the one or more patient data monitoring devices, the computing device including:
at least one processing device; and
at least one computer readable data storage device storing software instructions that, when executed by the at least one processing device, cause the at least one processing device to:
receive patient data from the one or more patient data monitoring devices;
analyze the patient data in real-time to learn one or more characteristics;
generate synthetic patient data based on the one or more characteristics, the synthetic patient data including data representative of the patient data that is different from the patient data; and
store the synthetic patient data in a database.

2. The system of claim 1, wherein the software instructions, when executed by the at least one processing device, further cause the at least one processing device to:
input the one or more characteristics of the patient data into an artificial intelligence algorithm to generate the synthetic patient data, the artificial intelligence algorithm stored in the at least one computer readable data storage device.

3. The system of claim 1, wherein the synthetic patient data is configured for use as training data for building a machine learning model.

4. The system of claim 1, wherein the one or more patient data monitoring devices include a thermal camera packaged together with the computing device in a single device.

5. The system of claim 4, wherein the software instructions, when executed by the at least one processing device, further cause the at least one processing device to:
receive a thermal image captured by the thermal camera; and
use the thermal image to generate the synthetic patient data.

6. The system of claim 5, wherein the synthetic patient data includes at least one of synthetic temperature measurements, synthetic respiration rate measurements, and synthetic patient position data.

7. The system of claim 1, wherein the wherein the software instructions, when executed by the at least one processing device, further cause the at least one processing device to:
generate the synthetic patient data based on patient data obtained from radar based contactless sensors.

8. The system of claim 1, wherein the wherein the software instructions, when executed by the at least one processing device, further cause the at least one processing device to:
generate a multimodal synthetic patent data set based on patient data obtained from a plurality of the patient data monitoring devices.

9. A method for generating synthetic patient data, the method comprising:
receiving patient data from one or more patient data monitoring devices;
analyzing the patient data in real-time to learn one or more characteristics;
generating synthetic patient data based on the one or more characteristics, the synthetic patient data including data representative of the patient data that is different from the patient data; and
storing the synthetic patient data in a database.

10. The method of claim 9, further comprising:
inputting the one or more characteristics of the patient data into an artificial intelligence algorithm to generate the synthetic patient data.

11. The method of claim 9, further comprising:
receiving a thermal image; and
using the thermal image to generate the synthetic patient data.

12. The method of claim 11, wherein the synthetic patient data includes at least one of synthetic temperature measurements, synthetic respiration rate measurements, and synthetic patient position data.

13. The method of claim 9, further comprising:
generating the synthetic patient data based on patient data obtained from radar based contactless sensors.

14. The method of claim 9, further comprising:
generating a multimodal synthetic patent data set based on patient data obtained from a plurality of the patient data monitoring devices.

15. The method of claim 9, wherein the synthetic patient data is configured for use as training data for building a machine learning model.

16. A non-transitory computer readable storage media including computer readable instructions which, when read and executed by a computing device, cause the computing device to:
receive patient data from one or more patient data monitoring devices;
analyze the patient data in real-time to learn one or more characteristics;
generate synthetic patient data based on the one or more characteristics, the synthetic patient data including data representative of the patient data that is different from the patient data; and
store the synthetic patient data in a database.

17. The non-transitory computer readable storage media of claim 16, further comprising computer readable instructions which when read and executed by the computing device, cause the computing device to:
learn one or more characteristics of the patient data; and
input the one or more characteristics of the patient data into an artificial intelligence algorithm to generate the synthetic patient data.

18. The non-transitory computer readable storage media of claim 16, further comprising computer readable instructions which when read and executed by the computing device, cause the computing device to:
generate the synthetic patient data based on patient data obtained from radar based contactless sensors.

19. The non-transitory computer readable storage media of claim 16, further comprising computer readable instructions which when read and executed by the computing device, cause the computing device to:
generate a multimodal synthetic patent data set based on patient data obtained from a plurality of the patient data monitoring devices.

20. The non-transitory computer readable storage media of claim 16, further comprising computer readable instructions which when read and executed by the computing device, cause the computing device to:
receive a thermal image captured by a thermal camera; and
use the thermal image to generate the synthetic patient data.
